(19) United States
(12) Patent Application Publication
(10) Pub. No.: US 2022/0073981 A1
(43) Pub. Date: Mar. 10, 2022

(54) INFLAMMATORY BIOMARKER SPECIFIC TO EXPOSURE TO 2-BUTANONE AND IDENTIFICATION METHOD USING SAME

(71) Applicant: KOREA INSTITUTE OF SCIENCE AND TECHNOLOGY, Seoul (KR)

(72) Inventors: Jae Chun RYU, Seoul (KR); Yoon CHO, Seoul (KR)

(73) Assignee: KOREA INSTITUTE OF SCIENCE AND TECHNOLOGY, Seoul (KR)

(21) Appl. No.: 17/466,881

(22) Filed: Sep. 3, 2021

(30) Foreign Application Priority Data
Sep. 7, 2020 (KR) 10-2020-0113655

(51) Int. Cl. C12Q 1/6876; C12Q 1/6874; C12N 15/10

(52) U.S. Cl. CPC C12Q 1/6876; C12Q 1/6874; C12Q 2600/158; C12Q 2600/142; C12N 15/1096

(57) ABSTRACT

The present invention provides a biomarker for identifying the expression of an inflammatory-response-associated gene that specifically causes a change in expression due to exposure to 2-butanone, which is harmful and found in indoor environments, and an identification method using the same, and particularly an inflammatory-response-associated gene, the expression of which is increased or decreased by exposure to 2-butanone in a human bronchial epithelial cell line model (BEAS-2B), and a method of identifying exposure and predicting an inflammatory response using the same. The biomarker of the present invention includes specific genes selected through RNA sequencing, and thus can be useful in monitoring and determining the exposure to 2-butanone in the environment, and can be utilized as a tool predicting the mechanism of inflammatory response and toxicity caused by exposure to 2-butanone.

### CROSS-REFERENCE TO RELATED APPLICATION

This application claims under 35 U.S.C. § 119 (a) the benefit of priority from Korean Patent Application No. 10-2020-0113655, filed on Sep. 7, 2020, the entire contents of which are incorporated herein by reference.

### BACKGROUND

### (a) Technical Field

The present disclosure relates to an inflammatory biomarker specific to exposure to 2-butanone and an identification method using the same, and more particularly to an inflammatory-response-associated biomarker, the gene expression of which is specifically increased or decreased by exposure to 2-butanone in a human bronchial epithelial cell line (BEAS-2B), and a method of identifying exposure to 2-butanone using the same.

### (b) Background Art

Recently, societal interest in human health problems caused by indoor air pollution is increasing. The World Health Organization (WHO) reports that the probability of indoor pollutants reaching the lungs is about 1,000 times higher than outdoor pollutants, and 4.2 million people die each year due to indoor air pollution, which is more than the number of people reported to die due to outdoor air pollution. Various pollutants that threaten the human body are continuously released indoors, where most modern people spend at least 80% of the time in daily lives, so systematic management through rational regulation therefor is necessary.

2-butanone (methyl ethyl ketone, CAS No. 78-93-3) is a colorless liquid, and has a structure similar to that of acetone. It is widely used in the manufacture of various detergents, ink additives, dye solvents, pigments, paints, adhesives, artificial leather, etc. and also as an intermediate for organic synthesis. 2-butanone is known to be a substance that causes severe irritation not only to the respiratory system but also to the eyes, skin, and the like when exposed thereto, and is reported to cause symptoms such as headaches, dizziness, vomiting, breathing difficulty and the like when inhaled through the respiratory system (Korpi A. Microbial Volatile Organic Compounds. Crit. Rev. Toxicol. 39 (2):139.193, 2009).

Despite the potential harmfulness of 2-butanone to human bodies as described above, particular exposure and risk assessment data thereof are not sufficient, and existing data are also limited to typical methods such as GC-MS (gas chromatography-mass spectrometry) or HPLC (high-performance liquid chromatography). Quantification is possible using GC-MS or HPLC methods, but appropriate conditions for analysis must be set, and expensive devices are required. Therefore, it is important to discover and utilize molecular indicators capable of searching for exposure and harmful effects in the human body and to perform appropriate countermeasures and control for exposure to 2-butanone through rapid risk assessment using real-time polymerase chain reaction, DNA microarray, RNA sequencing (RNA-Seq), etc.

RNA sequencing (RNA-Seq) is an analysis method that decodes the genome by dividing the genome into many fragments and combining nucleotide sequences. Subsequent to the human genome project in 2004, the nucleotide sequence information of an individual was decoded for the first time using the Sanger method (Levy et al., Nature 456: 2008), and then in 2008, due to the development of technology, decoding at 1% of the cost of initial nucleotide sequence information decoding technology became possible (Wheeler D A et al., Nature 452:2008). RNA sequencing is a method capable of analyzing the transcriptome with NGS technology and also of finding out various pieces of information about the transcriptome at once, including the expression of transcripts or the combinations of exons in the transcripts, by sequencing the RNA sequence expressed in a specific sample. In order to perform analysis comparing gene expression levels or to perform integrated analysis and interpretation of gene structures such as SNP, In/Del, and alternative splicing, RNA sequencing (RNA-seq) is carried out.

At present, research into toxic genomics, which is a cutting-edge technique using RNA sequencing (RNA-Seq) together with DNA microarrays, is being conducted, thus making it possible to analyze and quantitatively analyze the expression patterns of genes expressed in specific tissues or cell lines by all chemicals, including not only medicines and new medicine candidates but also representative environmental pollutants, with high throughput. Accordingly, the frequency of expression of specific genes in specific cells may be analyzed, making it possible to discover genes related to the side effects of drugs and harmful effects of environmental pollutants. Thereby, it will be possible to understand the molecular mechanisms due to the harmful actions of environmental pollutants and the actions and side effects of drugs, and also to accurately search for the materials that cause toxicity and side effects.

### CITATION LIST

### Non-Patent Literature

(Non-Patent Document 1) Korpi A. Microbial Volatile Organic Compounds. Crit. Rev. Toxicol. 39 (2):139.193, 2009

### SUMMARY OF THE DISCLOSURE

Accordingly, the present inventors observed and analyzed the gene expression profile of 2-butanone in a human bronchial epithelial cell line (BEAS-2B) using NextSeq500 (Illumina, Inc., USA), discovered inflammatory-response-associated biomarkers, the gene expression of which is specifically increased or decreased by exposure to 2-butanone, and established a method of identifying exposure to 2-butanone, thus culminating in the present invention.

Therefore, an object of the present invention is to provide an inflammatory-response-associated biomarker, the expression of which is specifically changed by exposure to 2-butanone, and a method of identifying exposure to 2-butanone using the biomarker.

In order to solve the above object, an aspect of the present invention provides a biomarker for identifying exposure to 2-butanone, which is an inflammatory-response-associated gene that specifically causes a change in expression due to exposure to 2-butanone, as described below:

Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

Another aspect of the present invention provides a method of identifying exposure to 2-butanone including 1) isolating total RNA from somatic cells of each of an experimental group exposed to 2-butanone and a normal control group, 2) hybridizing the total RNA of each of the experimental group and the control group isolated in step 1 with an oligo-dT primer, 3) subjecting the total RNA hybridized in step 2 to reverse transcription, 4) analyzing the nucleotide sequence of step 3 (high-throughput sequencing), and 5) identifying the expression level of the biomarker of the present invention through comparison with the control group based on data analyzed in step 4.

### BRIEF DESCRIPTION OF THE DRAWINGS

The above and other features of the present invention will now be described in detail with reference to certain exemplary embodiments thereof illustrated in the accompanying drawings which are given hereinbelow by way of illustration only, and thus are not limitative of the present invention, and wherein:

FIG. 1 shows the results of analysis of the expression pattern of genes, the expression of which is changed 2.0-fold or more in human bronchial epithelial cells (BEAS-2B) exposed to 2-butanone using RNA sequencing; and

FIG. 2 shows the results of analysis of the expression pattern of 13 types of inflammatory-response-associated genes among genes, the expression of which is specifically changed 2.0-fold or more in human bronchial epithelial cells (BEAS-2B) exposed to 2-butanone using RNA sequencing.

### DETAILED DESCRIPTION

Unless otherwise specified, all numbers, values, and/or representations that express the amounts of components, reaction conditions, polymer compositions, and mixtures used herein are to be taken as approximations including various uncertainties affecting measurement that inherently occur in obtaining these values, among others, and thus should be understood to be modified by the term “about” in all cases. Furthermore, when a numerical range is disclosed in this specification, the range is continuous, and includes all values from the minimum value of said range to the maximum value thereof, unless otherwise indicated. Moreover, when such a range pertains to integer values, all integers including the minimum value to the maximum value are included, unless otherwise indicated.

In the present specification, when a range is described for a variable, it will be understood that the variable includes all values within the stated range, including the end points. For example, the range of “5 to 10” will be understood to include any subranges, such as 6 to 10, 7 to 10, 6 to 9, 7 to 9 and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

Hereinafter, various aspects of the present invention will be described in detail.

An aspect of the present invention pertains to a composition for identifying exposure to 2-butanone, including at least one gene biomarker selected from the following group:

Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

Another aspect of the present invention pertains to a DNA microarray chip for identifying exposure to 2-butanone, in which at least one gene biomarker selected from the following group or a complementary strand molecule thereof is integrated: Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

Still another aspect of the present invention pertains to a kit for identifying exposure to 2-butanone including the DNA microarray chip for identifying exposure to 2-butanone.

In still another aspect of the present invention, the kit further includes human somatic cells.

Yet another aspect of the present invention pertains to a method of identifying exposure to 2-butanone including 1) isolating total RNA from a sample of each of an experimental group exposed to 2-butanone and a control group not exposed to 2-butanone, 2) hybridizing the total RNA of each of the experimental group and the control group isolated in step 1 with an oligo-dT primer, 3) subjecting the total RNA hybridized in step 2 to reverse transcription, 4) analyzing the nucleotide sequence of step 3 (high-throughput sequencing), and 5) identifying the expression level of the biomarker of the present invention through comparison with the control group based on the data analyzed in step 4.

In the method according to yet another aspect of the present invention, the sample of step 1 is human bronchial epithelial cells.

In the method according to yet another aspect of the present invention, the human bronchial epithelial cells are BEAS-2B.

Below is a detailed description of the present invention.

The present invention provides an inflammatory biomarker that specifically causes a change in expression due to exposure to 2-butanone.

The biomarker includes genes, the expression of which is increased or decreased 2.0-fold or more, particularly 13 types of inflammatory-response-associated genes, the expression of which is specifically changed by exposure to 2-butanone.

The biomarker that specifically causes a change in expression due to exposure to 2-butanone preferably includes the following genes, but is not limited thereto:

Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

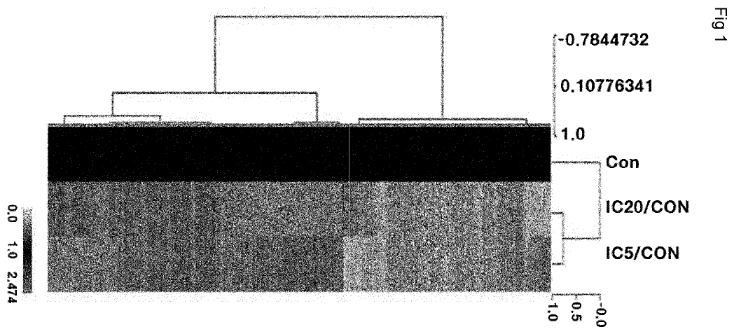

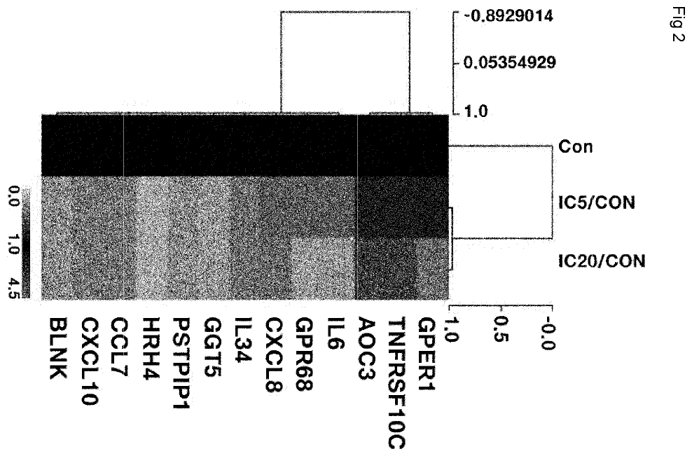

In a specific embodiment of the present invention, in order to search for a biomarker for identifying specific exposure to 2-butanone, based on the previously established in-vitro experimental technique, BEAS-2B, which is a human bronchial epithelial cell line, was exposed to 2-butanone, and total RNA was isolated from the BEAS-2B cells treated with the material described above. The total RNA thus isolated was subjected to hybridization with an oligo-dT primer and then reverse transcription, and the nucleotide sequence thereof was analyzed using NextSeq500 (Illumina, Inc., USA) (FIG. 1). The gene and expression level of mRNA were identified through sequencing. Among the genes of which the expression levels in the experimental group and the control group differed by 2.0-fold or more, the following 13 genes (3 overexpressed genes and 10 underexpressed genes) associated with inflammatory responses were selected (FIG. 2):

Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker)

Therefore, the biomarker of the present invention is a gene associated with an inflammatory response among genes, the expression of which is specifically increased or decreased by exposure to 2-butanone, and the biomarker is useful as a tool to confirm exposure to 2-butanone in the environment, perform monitoring, determine risk, and elucidate the mechanism of inflammatory response and pulmonary toxicity caused by 2-butanone.

In addition, the present invention provides a method of identifying gene expression due to exposure to 2-butanone using RNA sequencing, including:

1) isolating total RNA from the somatic cells of each of an experimental group exposed to 2-butanone and a normal control group;

2) hybridizing the total RNA of each of the experimental group and the control group isolated in step 1 with an oligo-dT primer;

3) subjecting the total RNA hybridized in step 2 to reverse transcription;

4) analyzing the nucleotide sequence of step 3 (high-throughput sequencing); and

5) identifying the expression level of the biomarker of the present invention through comparison with the control group based on the data analyzed in step 4.

In the method of identifying the exposure, the somatic cells of step 1 are preferably a human bronchial epithelial cell line, which may or may not be exposed to 2-butanone, but are not limited thereto, and any cell may be used therefor, so long as it is derived from tissue.

In the method, the human bronchial epithelial cells are preferably BEAS-2B, but are not limited thereto.

In the method of identifying the exposure, isolating the total RNA in step 2 is preferably performed using a TRIzol reagent, but the present invention is not limited thereto, and any method for isolation and purification of total RNA known to those skilled in the art may be used.

In the method of identifying the exposure, a single-end 75 sequencing method using NextSeq500 (Illumina, Inc., USA) is preferable for analyzing the nucleotide sequence in step 4, but the present invention is not limited thereto.

Moreover, for the analysis method of step 4, it is preferable to use GeneSpring GX 12.6.1 software (Agilent, USA), but the present invention is not limited thereto, and any analysis software known to those skilled in the art may be used.

The method of identifying gene expression according to the present invention uses an RNA sequencing method capable of identifying distribution of genes that are specifically overexpressed or underexpressed due to exposure to 2-butanone, and is useful as a tool to confirm exposure to 2-butanone in the environment, perform monitoring, determine risk, and elucidate the mechanism of inflammatory response and pulmonary toxicity caused by 2-butanone.

The present invention provides a kit for identifying exposure to 2-butanone in an environment including RNA sequencing.

The kit for identifying exposure to 2-butanone in the environment further includes human bronchial epithelial cells.

The human bronchial epithelial cells are preferably those obtained from a group exposed or not exposed to 2-butanone in the environment.

The kit may further include a reaction reagent, and examples of the reaction reagent may include, but are not limited to, a buffer for use in hybridization, oligo dT beads for isolating mRNA from total RNA, reverse transcriptase for cDNA synthesis, dNTP and rNTP (pre-mixed or separate supply type), ligase, washing buffer, and the like, and all reaction reagents required for mRNA hybridization and cDNA synthesis and amplification reactions for RNA-seq known to those skilled in the art may be included.

Therefore, the kit according to the present invention is useful as a tool to confirm exposure to 2-butanone in the environment, perform monitoring, determine risk, and elucidate the mechanism of inflammatory response and pulmonary toxicity caused by 2-butanone using the inflammatory-response-associated gene, the expression of which is specifically increased or decreased by exposure to 2-butanone.

A better understanding of the present invention may be obtained through the following preparation examples, examples, and experimental examples. These preparation examples, examples, and experimental examples are merely set forth to illustrate the present invention, and are not to be construed as limiting the present invention.

### <Example 1> Cell Culture and Chemical Treatment

<1-1> Cell Culture

As a human bronchial epithelial cell line, BEAS-2B (Korea Cell Line Bank), was cultured to about 70-80% confluence in a 100 mm dish using an RPMI 1640 medium (Gibco, USA) supplemented with 10% FBS. The present inventors selected 2-butanone, as one of indoor pollutants present in the environment, based on conventional studies and reports, and 2-butanone was dissolved in filtered D.W. The concentration of a vehicle was 0.1% or less in all experiments.

<1-2> Selection of Exposure Concentration and Chemical Treatment Through Cytotoxicity Test (MTT Assay)

An MTT assay was performed using the BEAS-2B cell line according to the method of Mossman et al. (J. Immunol. Methods, 65, 55-63, 1983).

Specifically, the cells were seeded at a density of 3.5×10⁴ cells/well in a 24-well plate and exposed to 2-butanone dissolved in filtered D.W. in an RPMI medium (Gibco, USA), and after 48 hours, the resulting cells were mixed with an MTT (3-4,5-dimethylthiazol-2,5-diphenyltetrazolium bromide) solution (5 mg/ml) and cultured at 37° C. for 3 hours. \acute{Ε}After the medium was removed, the formed formazan crystal was dissolved in 500 μl of DMSO, 100 μl thereof was transferred each time to a 96-well plate, and the optical density (OD) value thereof was measured at an absorbance 540 nm.

Based on the results of measurement of cytotoxicity of 2-butanone in the BEAS-2B cell line, the concentration (IC₅) showing the 95% survival rate was 22.27 mM, and the concentration (1020) showing the 80% survival rate was 106.21 mM. Based on these results, the following gene-sequencing experiment was performed.

### <Example 2> Gene-Sequencing Experiment

<2-1> Isolation of RNA

For RNA extraction from the cells of the group exposed to 2-butanone and the group not exposed to 2-butanone, total RNA was isolated according to the manufacturer's method using a TRIzol reagent (Life Technologies, USA) and purified using an RNeasy mini kit (Qiagen, USA). The concentration of each total RNA sample was determined using a spectrophotometer (ND-2000 Spectrophotometer, Thermo Inc., DE, USA) and an Agilent 2100 Bioanalyzer (Agilent Technologies, USA).

<2-2> Library Preparation

A library was prepared from total RNA obtained from each of the experimental group exposed to 2-butanone and the normal control group using a QuantSeq 3′ mRNA-Seq Library Prep Kit (Lexogen, Inc., Austria) according to the manufacturer's method. 500 ng of the total RNA and an oligo-dT primer were hybridized, followed by reverse transcription. The double-stranded library was purified using magnetic beads in order to remove all reaction components. The library was amplified in order to add the full adapter sequence required for cluster generation, and the completed library was purified from PCR components.

<2-3> Sequencing

Sequencing was performed through single-end 75 sequencing using a NextSeq 500 (IIlumina, Inc., USA). RC (Read Count) data was processed based on the quantile normalization method using EdgeR in R (R development Core Team, 2016) using a Bioconductor (Gentleman et al., 2004). Gene classification was based on a search performed by DAVID Bioinformatics Resources (http://david.ncifcrf.gov/).

The extracted data was normalized using an Agilent GeneSpring GX 12.6.1 (Agilent Technologies, CA, USA) to analyze the pattern of expression of each gene.

Based on the results thereof, among the genes, the expression of which was specifically changed 2.0-fold or more by exposure to 2-butanone (FIG. 1), there were 13 types of inflammatory-response-associated genes (Table 1 below and FIG. 2). There is no report that such genes are involved in toxicity upon exposure to 2-butanone in human bronchial epithelial cells.

TABLE 1
Inflammatory-response-associated genes specific to exposure to 2-butanone

| Gene | | Gene Accession | Fold Change | |
|---|---|---|---|---|
| Symbol | Gene Name | No. | IC₅ | IC₂₀ |
| IL6 | Interleukin 6 | NM_000600 | 0.480 | 0.243 |
| CXCL8 | C-X-C motif chemokine | NM_000584 | 0.496 | 0.421 |
| | ligand 8 | | | |
| GPER1 | G protein-coupled estrogen | NM_001039966 | 2.031 | 4.514 |
| | receptor 1 | | | |
| GPR68 | G protein-coupled receptor | NM_001177676 | 0.479 | 0.226 |
| | 68 | | | |
| IL34 | Interleukin 34 | NM_001172771 | 0.408 | 0.400 |
| CCL7 | C-C motif chemokine | NM_006273 | 0.376 | 0.374 |
| | ligand 7 | | | |
| CXCL10 | C-X-C motif chemokine | NM_001565 | 0.373 | 0.371 |
| | ligand 10 | | | |
| GGT5 | Gamma-glutamyltransferase 5 | NM_004121 | 0.247 | 0.243 |
| TNFRSF10C | Tumor necrosis factor | NM_003841 | 2.187 | 3.220 |
| | receptor superfamily | | | |
| | member 10c | | | |
| HRH4 | Histamine receptor H4 | NM_001160166 | 0.167 | 0.165 |
| PSTPIP1 | Proline-serine-threonine | NM_003978 | 0.288 | 0.285 |
| | phosphatase interacting | | | |
| | protein 1 | | | |
| AOC3 | Amine oxidase | NM_003734 | 2.207 | 3.241 |
| | copper-containing 3 | | | |
| BLNK | B-cell linker | NR_047682 | 0.286 | 0.284 |

As is apparent from the above description, an inflammatory biomarker specific to exposure to 2-butanone according to the present invention and an identification method using the same are useful for monitoring exposure to 2-butanone and determining the risk thereof using, as a biomarker, an inflammatory-response-associated gene selected through RNA sequencing, and can be utilized in elucidating the mechanism of inflammatory response and toxicity caused by 2-butanone.

Although specific embodiments of the present disclosure have been described with reference to the accompanying drawings, those skilled in the art will appreciate that the present disclosure may be embodied in other specific forms without changing the technical spirit or essential features thereof. Thus, the embodiments described above should be understood to be non-limiting and illustrative in every way.

What is claimed is:

1. A composition for identifying exposure to 2-butanone, comprising at least one gene biomarker selected from a following group:
Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

2. A DNA microarray chip for identifying exposure to 2-butanone, in which at least one gene biomarker selected from a following group or a complementary strand molecule thereof is integrated:
Gene Accession No. (GenBank) NM_000600 (IL6, Interleukin 6), Gene Accession No. (GenBank) NM_000584 (CXCL8, C-X-C motif chemokine ligand 8), Gene Accession No. (GenBank) NM_001039966 (GPER1, G protein-coupled estrogen receptor 1), Gene Accession No. (GenBank) NM_001177676 (GPR68, G protein-coupled receptor 68), Gene Accession No. (GenBank) NM_001172771 (IL34, Interleukin 34), Gene Accession No. (GenBank) NM_006273 (CCL7, C-C motif chemokine ligand 7), Gene Accession No. (GenBank) NM_001565 (CXCL10, C-X-C motif chemokine ligand 10), Gene Accession No. (GenBank) NM_004121 (GGT5, Gamma-glutamyltransferase 5), Gene Accession No. (GenBank) NM_003841 (TNFRSF10C, Tumor necrosis factor receptor superfamily member 10c), Gene Accession No. (GenBank) NM_001160166 (HRH4, Histamine receptor H4), Gene Accession No. (GenBank) NM_003978 (PSTPIP1, Proline-serine-threonine phosphatase interacting protein 1), Gene Accession No. (GenBank) NM_003734 (AOC3, Amine oxidase copper-containing 3), and Gene Accession No. (GenBank) NR_047682 (BLNK, B-cell linker).

3. A kit for identifying exposure to 2-butanone, comprising the DNA microarray chip of claim 2.

4. The kit of claim 3, further comprising human somatic cells.

5. A method of identifying exposure to 2-butanone, comprising:
1) isolating total RNA from a sample of each of an experimental group exposed to 2-butanone and a control group not exposed to 2-butanone;
2) hybridizing the total RNA of each of the experimental group and the control group isolated in step 1 with an oligo-dT primer;
3) subjecting the total RNA hybridized in step 2 to reverse transcription;
4) analyzing a nucleotide sequence of step 3 (high-throughput sequencing); and
5) identifying an expression level of the biomarker of claim 1 through comparison with the control group based on data analyzed in step 4.

6. The method of claim 5, wherein the sample of step 1 is human bronchial epithelial cells.

7. The method of claim 6, wherein the human bronchial epithelial cells are BEAS-2B.
